(19) United States
(12) Patent Application Publication
(10) Pub. No.: US 2024/0226503 A1
(43) Pub. Date: Jul. 11, 2024

(54) CATHETER TIP SHAPING TOOL

(71) Applicant: Boston Scientific Medical Device Limited, Galway (IE)

(72) Inventors: Rahul Jain, Meerut / Uttar Pradesh (IN); Prateek Gupta, Gurgaon / Haryana (IN); Shoeb Khan, Gurgaon / Haryana (IN); Jahnavi Konduru, New Delhi / Delhi (IN); Anshul Chabra, Delhi (IN)

(73) Assignee: Boston Scientific Medical Device Limited, Galway (IE)

(21) Appl. No.: 18/409,144

(22) Filed: Jan. 10, 2024

Related U.S. Application Data
(60) Provisional application No. 63/438,317, filed on Jan. 11, 2023.

(51) Int. Cl. A61M 25/00

(52) U.S. Cl. CPC A61M 25/0068; A61M 2207/10

(57) ABSTRACT

A catheter tip shaping system includes a catheter, and a tip shaping tool. The tip shaping tool includes a body having a first end and a second end, a first pin, a second pin, and a third pin, wherein the first, second, and third pins are positioned at the first end of the body and extend from a first face of the body. A distal end region of the catheter may be configured to be positioned between a gap between the first, second, and third pins such that the tip shaping tool can be manipulated by a user to invoke a curvature in the distal end region of the catheter.

### CROSS-REFERENCE TO RELATED APPLICATIONS

This application claims the benefit of U.S. Provisional Patent Application Ser. No. 63/438,317, filed Jan. 11, 2023, which is incorporated herein by reference.

### TECHNICAL FIELD

The present disclosure pertains to catheters for delivery of therapeutic agents or devices to a site within a body lumen, and associated accessories. More particularly, the present disclosure pertains to catheters and tip shaping tools for catheters.

### BACKGROUND

A variety of intravascular catheters are known, including small diameter catheters having a central lumen therethrough that are configured for use in smaller vasculature. Such catheters are known as microcatheters. Microcatheters are typically highly flexible and thin-walled, which can result in limited torque transfer from the proximal hub to the distal tip, reduced kink resistance, and difficulty pushing through tortuous vasculature. Typically, a tip of the microcatheter requires shaping to navigate such tortuous vasculature. Current methods include steam-shaping and hand-shaping. However, these methods are time-consuming, lack precision of the desired angle, and may result in a damaged tip. A need remains for improved catheter tip shaping tools and methods that decrease time consumption and damage, while increasing torque transfer and kink resistance.

There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a catheter tip shaping system which may include a catheter, and a tip shaping tool. The tip shaping tool may include a body having a first end and a second end, a first pin, a second pin, and a third pin, wherein the first, second, and third pins are positioned at the first end of the body and extend from a first face of the body. A distal end region of the catheter may be configured to be positioned between a gap between the first, second, and third pins such that the tip shaping tool can be manipulated by a user to invoke a curvature in the distal end region of the catheter.

Alternatively, or additionally to any of the embodiments above, the first, second, and third pins may be positioned equidistant from one another.

Alternatively, or additionally to any of the embodiments above, a distance between each of the first, second, and third pins may be about 0.025 inches to about 0.045 inches.

Alternatively, or additionally to any of the embodiments above, the body may include a grip portion to be grasped by the user.

Alternatively, or additionally to any of the embodiments above, the first, second, and third pins may each include a circular cross-section.

Alternatively, or additionally to any of the embodiments above, the first, second, and third pins may each include a first end region and a second end region, wherein the first end region includes a first outer diameter and the second end region includes a second outer diameter, and the first outer diameter is greater than the second outer diameter.

Alternatively, or additionally to any of the embodiments above, the second end regions of the first, second, and third pins may be coupled to the first end of the body of the tip shaping tool with the second end regions of the first, second, and third pins positioned between the first face of the body and the first end regions of the first, second and third pins.

Alternatively, or additionally to any of the embodiments above, a gap between the first end regions of the first, second, and third pins may be less than an outer diameter of the distal end region of the catheter and a gap between the second end regions of the first, second, and third pins is greater than the outer diameter of the distal end region of the catheter.

Alternatively, or additionally to any of the embodiments above, a length of the second end regions of the first, second, and third pins may be greater than an outer diameter of the distal end region of the catheter.

Alternatively, or additionally to any of the embodiments above, the first pin may be positioned on a first side of the distal end region of the catheter and the second and third pins are positioned on an opposite, second side of the distal end region of the catheter when the distal end region of the catheter is positioned in the gap.

Alternatively, or additionally to any of the embodiments above, the body may be configured to be rotated to invoke the curve in the distal end region.

Alternatively, or additionally to any of the embodiments above, the first pin may be located on a concave side of the curve, and the second and third pins are located on a convex side of the curve.

Alternatively, or additionally to any of the embodiments above, the catheter may include an elongated shaft having a distal end and a proximal end. The elongated shaft may include an outer layer formed of a polymer, an inner layer formed of a polymer, and a middle layer formed of a braid having a plurality of strands. Each strand may include a plurality of metal filaments, such as at least two, at least three, or at least four metal filaments each, for example. A polymeric distal tip may be attached to the distal end of the elongated shaft.

A method of shaping a distal end of a catheter using a tip shaping tool may include inserting a distal end region of the catheter in a gap between a first, a second, and a third pin of the tip shaping tool. The tip shaping tool may include a body having a first end and a second end, wherein the first pin, the second pin, and the third pin may be positioned at the first end of the body and extend from a first face of the body. The method may further include rotating the tip shaping tool such that the distal end region of the catheter moves from a first, straight configuration to a second, curved configuration, and removing the distal end region of the catheter from the tip shaping tool, wherein the tip shaping tool can be rotated in a clockwise direction and/or a counter-clockwise position.

Alternatively, or additionally to any of the embodiments above, when the distal end of the catheter is inserted into the gap of the tip shaping tool, the first pin may be located on a first side of the distal end region of the catheter, and the second and third pins are positioned on an opposite, second side of the distal end region of the catheter.

Alternatively, or additionally to any of the embodiments above, the first pin may be located on a concave side of the distal end region in the second, curved configuration, and the second and third pins are located on a convex side of the distal end region in the second, curved configuration.

Alternatively, or additionally to any of the embodiments above, when the distal end region of the catheter is inserted into the tip shaping tool, an end of a marker band positioned on the catheter may be aligned with an edge of the first end of the tip shaping tool prior to rotating the tip shaping tool.

In another example, a catheter tip shaping tool may include a body having a first end and a second end, and a first pin, a second pin, and a third pin, wherein the first, second, and third pins are positioned at the first end of the body and extend from a first face of the body.

Alternatively, or additionally to any of the embodiments above, the first, second, and third pins may be positioned equidistant from one another.

Alternatively, or additionally to any of the embodiments above, the first, second, and third pins may each include a first end region and a second end region, wherein the first end region is positioned between the first face of the body and the second end region, and the first end region includes a smaller outer diameter than an outer diameter of the second end region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of a tip shaping tool;

FIG. 2 is a side view of the tip shaping tool of FIG. 1;

FIG. 3 is an end view of the tip shaping tool of FIG. 1;

FIG. 4 is a perspective view of a tip shaping tool;

FIG. 5 is a side view of the tip shaping tool of FIG. 4;

FIG. 6 is an end view of the tip shaping tool of FIG. 4;

FIG. 7 is an enlarged view of a first end of the tip shaping tool as in FIG. 5, including a catheter;

FIGS. 8A to 8B depict an illustrative method of using a tip shaping tool with a catheter;

FIG. 9 depicts an illustrative graph comparing tip angle retention between a catheter tip shaped using a tip shaping tool in accordance with the disclosure versus a pre-shaped catheter tip;

FIG. 10 is a partial view of an elongated shaft of a catheter in accordance with an embodiment of the disclosure, illustrating an outer layer, a middle layer, and an inner layer of the elongated shaft;

FIG. 11 is a partial view of a braid of an elongated shaft of a catheter in accordance with an embodiment of the disclosure; and

FIG. 12 depicts a kit including a catheter and a tip shaping tool.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification. All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure. The recitation of numerical ranges by endpoints includes all numbers within that range (e.g., 1 to 5 includes, 1, 1.5, 2, 2.75, 3, 3.80, 4, and 5). As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the claims.

Catheters are typically highly flexible and thin-walled, which can result in limited torque transfer from proximal hub to distal tip, reduced kink resistance, and difficulty pushing through tortuous vasculature. Further, often times, a tip of the catheter requires shaping to navigate such tortuous vasculature. Other medical devices, such as guidewires, microcatheters, and the like, may also require tip shaping prior to use. Current methods of shaping may include steam-shaping and hand-shaping. However, these methods may be time-consuming, and may result in a damaged tip. The ability to shape a medical device without the use of steam may be desirable.

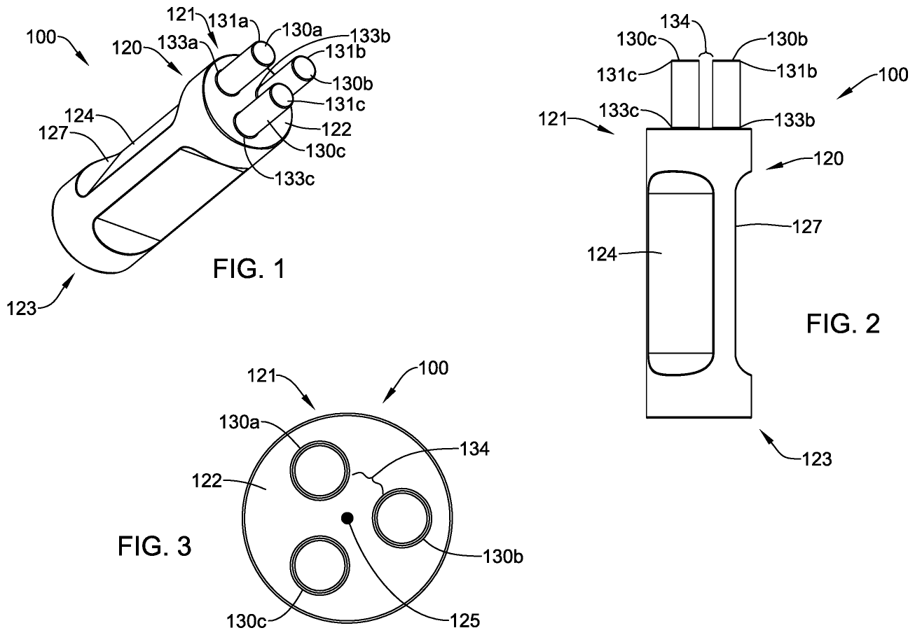

FIG. 1 is a perspective view of a tip shaping tool 100, FIG. 2 is a side view of the tip shaping tool 100 of FIG. 1, and FIG. 3 is an end view of the tip shaping tool 100 of FIG. 1. The tip shaping tool 100 may include a body 120 having a first end 121 and a second end 123. The tip shaping tool 100 may have any desired length and diameter. For example, the tip shaping tool 100 may have a length that is in the range of about 2 centimeters to 8 centimeters, and may have an outer diameter (OD) that is about 0.6 centimeters to 1.5 centimeters, for example. The tip shaping tool 100 may be formed from a rigid material, such as polymeric and/or metallic materials (e.g., acrylonitrile butadiene styrene, thermoplastics, other polymers, glass, metal, alloy, ceramic, and the like). Other embodiments may however, include tip shaping tools 100 made from a flexible or semi-rigid material, such as flexible or semi-rigid plastic materials, or any other suitable type of material, such as those disclosed further herein. Further, the tip shaping tool 100 may be formed via any suitable manufacturing technique including extruding, co-extruding, molding, casting, 3-D printing, mechanical working, and the like.

The body 120 of the tip shaping tool 100 may have any desired shape or geometry. For example, the body 120 may include a generally cylindrical shape, as indicated in FIGS. 1 to 3. In some cases, the body 120 of the tip shaping tool 100 may include a rectangular shape, a square shape, an oval shape, or any other suitable shape, as desired. The body 120 may further include a grip portion 124 positioned between the first end 121 and the second end 123. The grip portion 124 may be configured to be grasped by a user during use. For example, the grip portion 124 may include one or more depressions 127 that may include a shape and size configured for a user's thumb and/or fingers. In some cases, the grip portion 124 may not include the one or more depressions 127 and rather the body 120 may include one or more protrusions, flat surfaces, or the body 120 may include an uninterrupted cylindrical shape, or any other shape as desired.

A plurality of pins may extend from the first end 121 of the body 120. For example, a first pin 130a, a second pin 130b, and a third pin 130c may be positioned at the first end 121 of the body 120, and may extend from a first face 122 of the body 120. The first pin 130a, the second pin 130b, and the third pin 130c each include a first end region 131a, 131b, 131c, respectively, and a second end region 133a, 133b, 133c, respectively. The second end regions 133a, 133b, 133c may be coupled to the first end 121 of the body 120 of the tip shaping tool 100 with the second end regions 133a, 133b, 133c positioned between the first face 122 of the body 120 and the first end regions 131a, 131b, 131c. As shown in FIGS. 1 to 3, the first pin 130a, the second pin 130b, and the third pin 130c may each include a circular cross-section. However, in other instances, one or more of the first pin 130a, the second pin 130b, and/or the third pint 130c may include a different cross-sectional shape. For example, while it is shown that the first pin 130a, the second pin 130b, and the third pin 130c are shown to include a circular cross-section, it may be contemplated that the first pin 130a, the second pin 130b, and the third pin 130c may include a rectangular cross-section, a square cross-section, a triangular cross-section, or any other suitable cross-section. In some cases, the first pin 130a may include a circular cross-section, and the second pin 130b and the third pin 130c may include a cross-sectional shape different than that of the first pin 130a. In some cases, a side surface of one or more of the first pin 130a, the second pin 130b, and/or the third pint 130c may be a convex or arcuate surface. In some cases, it may be contemplated that the first pin 130a, the second pin 130b, and the third pin 130c may each include a cross-sectional shape different than one another. These are just examples.

In some cases, the first pin 130a, the second pin 130b, and the third pin 130c may be positioned equidistant from one another. For example, as shown in FIG. 3, the body 120 may include a central rotational axis 125, and the first pin 130a, the second pin 130b, and the third pin 130c may each be positioned equidistantly from one another around the central rotational axis 125. Furthermore, the first pin 130a, the second pin 130b, and the third pin 130c may each be positioned equidistantly from the central rotational axis 125. In some cases, the first pin 130a, the second pin 130b, and the third pin 130c may not be equidistant from one another and/or one or more of the first pin 130a, the second pin 130b, and the third pin 130c may be positioned further from the central rotational axis 125 than another one of the first pin 130a, the second pin 130b, and the third pin 130c. The distance between each of the first pin 130a, the second pin 130b, and the third pin 130c may include a distance configured to accommodate a catheter positioned therebetween. In some cases, a gap 134 between the first pin 130a, the second pin 130b, and the third pin 130c may include an equal distance between the first end regions 131a, 131b, 131c and the second end regions 133a, 133b, 133c. The pins 130a, 130b, 130c may be positioned such that the distance between each of the first pin 130a, the second pin 130b, and the third pin 130c may be slightly larger than the outer diameter of an associated catheter. For example, in some cases, the catheter may be a microcatheter, and the distance between each of first pin 130a, the second pin 130b, and the third pin 130c may include a distance of about 0.025 inches to about 0.045 inches. In some cases, the distance between the first pin 130a, the second pin 130b, and the third pin 130c may include a distance of about 0.04 inches to about 0.06 inches. In some cases, the distance between the first pin 130a, the second pin 130b, and the third pin 130c may be about 0.03 inches. These are just examples.

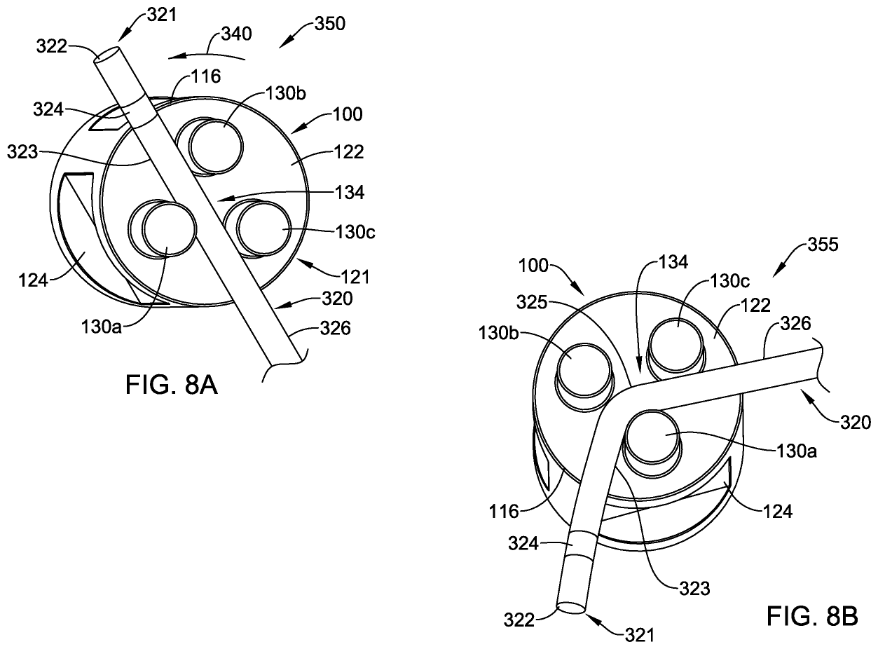

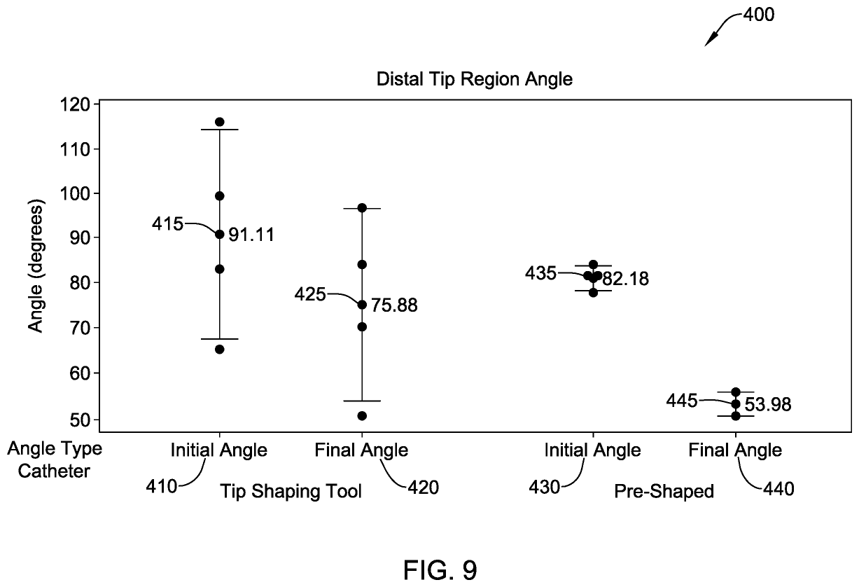

In some cases, in use, the first pin 130a may be positioned on a first side of a distal end region of a catheter, and the second pin 130b and the third pin 130c may be positioned on an opposite, second side of the distal end region of the catheter when the distal end region of the catheter is positioned in the gap 134, as further described with reference to FIGS. 8A to 8B. When the catheter is positioned in the gap 134, the body 120 of the tip shaping tool 100 may be configured to be manipulated, e.g., rotated about the central rotational axis 125, to invoke a curve in the distal end region of the catheter. In some cases, the tip shaping tool 100 may be rotated in a clockwise position. In some cases, the tip shaping tool 100 may be rotated in a counter-clockwise position.

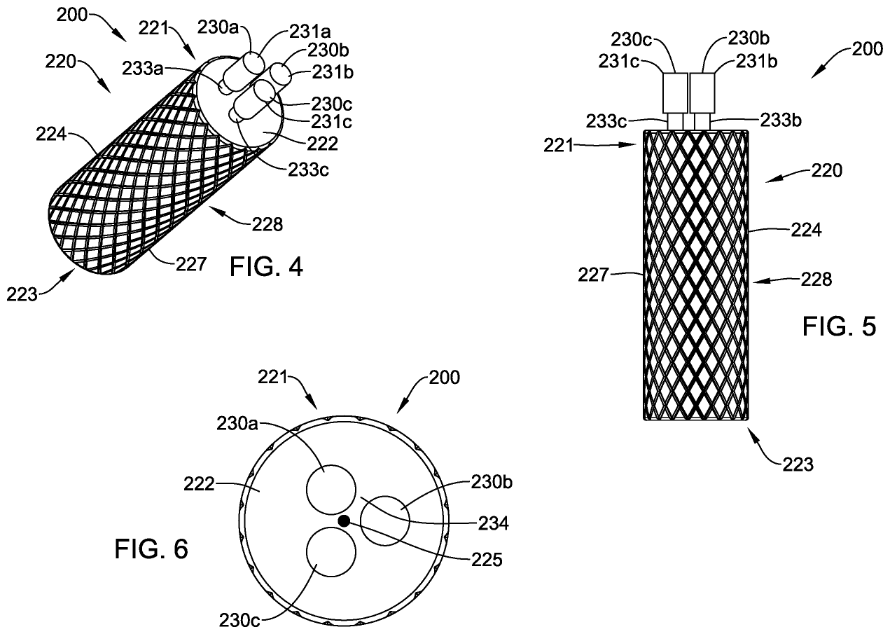

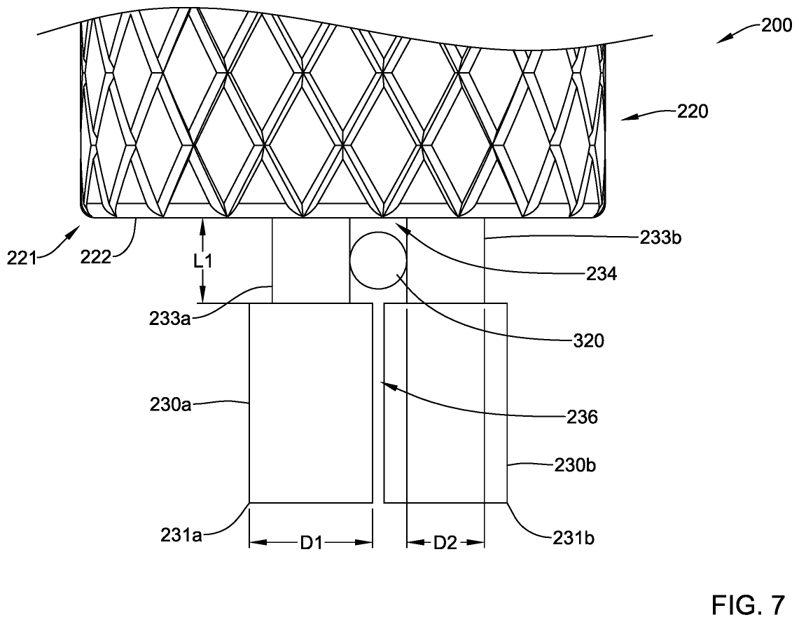

FIG. 4 is a perspective view of a tip shaping tool 200, FIG. 5 is a side view of the tip shaping tool 200 of FIG. 4, and FIG. 6 is a top view of the tip shaping tool 200 of FIG. 4. In many respects, the tip shaping tool 200 may be similar to the tip shaping tool 100, described above. For example, the tip shaping tool 200 may include a body 220 having a first end 221 and a second end 223. The tip shaping tool 200 may have any desired length and diameter. For example, the tip shaping tool 200 may have a length that is in the range of about 2 centimeters to 8 centimeters, and may have an outer diameter (OD) that is about 0.6 centimeters to 1.5 centimeters, for example. The tip shaping tool 200 may be formed from a rigid material, such as polymeric and/or metallic materials (e.g., acrylonitrile butadiene styrene, thermoplastics, other polymers, glass, metal, alloy, ceramic, and the like). Other embodiments may however, include tip shaping tools 200 made from a flexible or semi-rigid material, such as flexible or semi-rigid plastic materials, or any other suitable type of material, such as those disclosed further herein. Further, the tip shaping tool 200 may be formed via any suitable manufacturing technique including extruding, co-extruding, molding, casting, 3-D printing, mechanical working, and the like.

The body 220 of the tip shaping tool 200 may have any desired shape or geometry. For example, the body 220 may include a generally cylindrical shape, as indicated in FIGS. 4 to 6. In some cases, the body 220 of the tip shaping tool 200 may include a rectangular shape, a square shape, an oval shape, or any other suitable shape, as desired. The body 220 may further include a grip portion 224 positioned between the first end 221 and the second end 223. The grip portion 224 may be configured to be grasped by a user during use. For example, the grip portion 224 may include a roughened pattern 227, such as an intersecting grooved pattern or other knurled surface pattern, that may extend along the length of the body 220, as shown in FIGS. 4 to 5. In some cases, the roughened pattern 227 may be at the first end 221, the second end 223, or a medial region 228, or any combination thereof. In other instances, the grip portion 224 may be another configuration, such as but not limited to, the depressions described above regarding the tip shaping tool 100.

A plurality of pins may extend from the first end 221 of the body 220. For example, a first pin 230a, a second pin 230b, and a third pin 230c may be positioned at the first end 221 of the body 220, and may extend from a first face 222 of the body 220. The first pin 230a, the second pin 230b, and the third pin 230c each include a first end region 231a, 231b, 231c, respectively, and a second end region 233a, 233b, 233c, respectively. The second end regions 233a, 233b, 233c may be coupled to the first end 221 of the body 220 of the tip shaping tool 200 with the second end regions 233a, 233b, 233c positioned between the first face 222 of the body 220 and the first end regions 231a, 231b, 231c. The first end regions 231a, 231b, 231c may each include a first outer diameter (e.g., D₁, as shown in FIG. 7), and the second end regions 233a, 233b, 233c may each include a second outer diameter (e.g., D₂, as shown in FIG. 7). In some cases, the first outer diameter D₁ may be greater than the second outer diameter D₂, as shown in FIGS. 4, 5, and 7. In some cases, the first outer diameter D₁ may be the same as the second outer diameter D₂.

As shown in FIGS. 4 to 6, the first pin 230a, the second pin 230b, and the third pin 230c may each include a circular cross-section. However, in other instances, one or more of the first pin 230a, the second pin 230b, and/or the third pint 230c may include a different cross-sectional shape. For example, while it is shown that the first pin 230a, the second pin 230b, and the third pin 230c are shown to include a circular cross-section, it may be contemplated that the first pin 230a, the second pin 230b, and the third pin 230c may include a rectangular cross-section, a square cross-section, a triangular cross-section, or any other suitable cross-section. In some cases, the first pin 230a may include a circular cross-section, and the second pin 230b and the third pin 230c may include a cross-sectional shape different than that of the first pin 230a. In some cases, a side surface of the second end region 231a, 231b, 231c, of one or more of the first pin 130a, the second pin 130b, and/or the third pint 130c may be a convex or arcuate surface. In some cases, it may be contemplated that the first pin 230a, the second pin 230b, and the third pin 230c may each include a cross-sectional shape different than one another. These are just examples.

In some cases, the first pin 230a, the second pin 230b, and the third pin 230c may be positioned equidistant from one another. For example, as shown in FIG. 6, the body 220 may include a central rotational axis 225, and the first pin 230a, the second pin 230b, and the third pin 230c may each be positioned equidistantly from one another around the central rotational axis 225. Furthermore, the first pin 230a, the second pin 230b, and the third pin 230c may each be positioned equidistantly from the central rotational axis 225. In some cases, the first pin 230a, the second pin 230b, and the third pin 230c may not be equidistant from one another and/or one or more of the first pin 230a, the second pin 230b, and the third pin 23c may be positioned further from the central rotational axis 225 than another one of the first pin 230a, the second pin 120b, and the third pin 230c. The distance between each of the second end regions 233a, 233b, 233c of the first pin 230a, the second pin 230b, and the third pin 230c, respectively, may include a distance configured to accommodate a catheter positioned therebetween, as shown in FIG. 7. The pins 230a, 230b, 230c may be positioned such that the distance between each of the second end region 233a of the first pin 130a, the second end region 233b of the second pin 130b, and the second end region 233c of the third pin 130c may be slightly larger than the outer diameter of an associated catheter. For example, in some cases, the catheter may be a microcatheter, and the distance between each of the second end regions 233a, 233b, 233c may include a distance of about 0.025 inches to about 0.045 inches. In some cases, the distance between each of the second end regions 233a, 233b, 233c may include a distance of about 0.04 inches to about 0.06 inches. In some cases, the distance between each of the second end regions 233a, 233b, 233c may be about 0.03 inches. These are just examples.

In some cases, in use, the first pin 230a may be positioned on a first side of a distal end region of a catheter, and the second pin 230b and the third pin 230c may be positioned on an opposite, second side of the distal end region of the catheter when the distal end region of the catheter is positioned in the gap 234, as further described with reference to FIGS. 8A to 8B. When the catheter is positioned in the gap 234, the body 220 of the tip shaping tool 200 may be configured to be manipulated, e.g., rotated about the central rotational axis 225, to invoke a curve in the distal end region of the catheter. In some cases, the tip shaping tool 200 may be rotated in a clockwise position. In some cases, the tip shaping tool 200 may be rotated in a counter-clockwise position.

FIG. 7 is an enlarged view of the first end 221 of the tip shaping tool 200 as in FIG. 5, including a catheter 320. As shown in FIG. 7, the catheter 320 is positioned within the gap 234 between the first pin 230a (positioned on a first side of the distal end region of the catheter 320), the second and third pins 230b, 230c (positioned on a second, opposite side of the distal end region of the catheter 320).

The third pin 230c is not shown in the side view in FIG. 7. As can be seen, the first pin 230a, the second pin 230b (and the third pin 230c, although not shown explicitly), include a step, wherein the first end regions 231a, 231b, 231c include a first, outer diameter D₁ that is greater than a second, outer diameter D₂ of the second end regions 233a, 233b, 233c. In some cases, the first outer diameter D₁ may be in a range of about 0.05 centimeters to about 1.0 centimeters, and the second outer diameter D₂ may in a range of about 0.03 to about 0.08 centimeters. These are just examples. In some cases, a gap 236 between the first end regions 231a, 231b, 231c may be less than the outer diameter of the distal end region of the catheter 320, while the gap 234 between the second end regions 233a, 233b, 233c may be greater than the outer diameter of the distal end region of the catheter 320. Thus, the gap 234 between the second end regions 233a, 233b, 233c may be greater than a gap 236 between the first end regions 231a, 231b, 231c. In some cases, the second end regions 233a, 233b, 233c may include a length L that may be greater than the outer diameter of the distal end region of the catheter 320. In such cases, the distal end region of the catheter 320 may fit within the gap 234 between the second end regions 233a, 233b, 233c, but may not fit within the gap 236 between the first end regions 231a, 231b, 231c. Therefore, the first end regions 231a, 231b, 231c may serve to hold the distal end region of the catheter 320 within the gap 234 between the second end regions 233a, 233b, 233c while the user is shaping the tip of the catheter 320 with the tip shaping tool 200. In some cases, the length L may be in a range of about 0.03 inches to about 0.05 inches, or more, or in the range of about 0.05 inches to about 0.07 inches, or more. This is just an example. Thus, the distal end region of the catheter 320 may be advanced into the gap 234 between the second end region 233a of the first pin 230a and the second end regions 233b, 233c of the second and third pins 230b, 230c in a direction substantially perpendicular to the rotational axis 225, while the first end regions 231a, 231b, 231c of the first, second and third pins 230a, 230b, 230c prevent removal of the distal end region of the catheter 320 from the gap 234 in a direction substantially parallel to the rotational axis 225.

FIGS. 8A to 8B depict an illustrative method of using the tip shaping tool 100 with a catheter 320. Although the method is described using the tip shaping tool 100, it is noted that the tip shaping tool 200 may be used to shape the distal end region of the catheter 320 in a similar fashion. The method may include a user grasping the tip shaping tool 100 at the grip portion 124 of the tip shaping tool 100, and inserting a distal end region 321 of the catheter 320 in the gap 134 between the first pin 130a and the second and third pins 330b, 330c of the tip shaping tool 100. In some cases, the user may insert the distal end region 321 through the gap 134 with the distalmost tip 322 of the distal end region 321 beyond a peripheral edge 116 of the first end 121 of the body 120 of the tip shaping tool 100 according to a desired throw length. For example, in some cases, the distal end region 321 may be inserted such that the distalmost tip 322 of the distal end region 321 extends beyond the peripheral edge 116 in a range of about 0.1 centimeters to about 0.5 centimeters, for example. In some cases, the distal end region 321 of the catheter 320 may include a marker band 324. When the catheter 320 includes the marker band 324, the distal end region 321 of the catheter 320 may be inserted into the tip shaping tool 310 such that an edge (e.g., a proximal end or distal end) of the marker band 324 is aligned with the peripheral edge 116 of the first end 121 of the tip shaping tool 100, prior to rotating the body 120 of the tip shaping tool 100.

In some cases, the first pin 130a may be located on a first side 323 of the distal end region 321 of the catheter 320, while the second pin 130b and the third pin 130c both are positioned on an opposite, second side 325 of the distal end region 321 of the catheter 320. While it is illustrated that the first pin 130a is located at the first side 323 of the distal end region 321, and the second pin 130b and the third pin 130c are located on the second side 325 of the distal end region 321, it may be contemplated that the second pin 130b may be located at the first side 323 of the distal end region 321, and the first pin 130a and the third pin 130c may be located on the second side 325 of the distal end region 321. It may also be contemplated that the third pin 130c may be located at the first side 323 of the distal end region 321, and the second pin 130b and the first pin 130a may be located on the second side 325 of the distal end region 321. In other words, one of the first, second, and third pins 130a, 130b, 130c may be positioned on the first side 323 of the distal end region 321 of the catheter 320 while the other two of the first, second, and third pins 130a, 130b, 130c may be positioned on the second side of the distal end region 321 of the catheter 320.

With the distal end region 321 positioned in the gap 134, the tip shaping tool 310 may be rotated about the central rotational axis 125 (shown in FIG. 3) such that the distal end region 321 of the catheter 320 moves from a first, straight configuration 350 (shown in FIG. 8A) to a second, curved configuration 355 (shown in FIG. 8B). In some cases, when the catheter 320 has moved from the first, straight configuration 350 to the second, curved configuration 355, it may be considered that the first pin 130a is located on a concave side (e.g., the first side 323) and the second pin 130b and the third pin 130c are both located on a convex side (e.g., the second side 325) of the distal end region 321. As discussed above, if the distal end region 321 were inserted between the pins 130a, 130b, 130c in a different orientation, it may be contemplated that the second pin 130b may be located at the concave side of the distal end region 321, and the first pin 130a and the third pin 130c may be located on the convex side of the distal end region 321, or the third pin 130c may be located at the concave side of the distal end region 321, and the second pin 130b and the first pin 130a may be located on the convex side of the distal end region 321. While it is illustrated that the tip shaping tool 100 is rotated in a counter-clockwise direction, as indicated by arrow 340, it may be considered that the tip shaping tool 310 may also be rotated in a clockwise direction. The tip shaping tool 100 may be rotated to invoke curvature in the distal end region 321 of the catheter 320 to any degree desired for navigating through various lumens within a body. For example, in some cases, the tip shaping tool 100 may be rotated to invoke a 45° (degree) angle in the distal end region 321, a 55° angle, a 60° angle, a 90° angle, or any other suitable degree angle. The angle may provide a bend between the distal end region 321 and a proximal portion 326 of the catheter 320 proximal of the bend such that the distal end region 321 is not parallel to the proximal portion 326. The invoked angle may be dictated by the amount the tip shaping tool 100 is rotated. Once the desired angle is invoked in the distal end region 321, the distal end region 321 of the catheter 320 may be removed from the tip shaping tool 100.

The above-described method of shaping the distal tip region 321 of the catheter 320 may be formed with the catheter 320 at room temperature at the location of the surgical procedure (e.g., operating room, emergency room, catheter lab, etc.), without applying heat from a heating source (e.g., without applying steam, etc.) Furthermore, the above-described method of shaping the distal tip region 321 of the catheter 320 may be performed manually by the medical personnel during a medical procedure (i.e., intra-operatively) upon removing the catheter 320 from its packaging. As noted below, in some instances the tip shaping tool 100, 200 may be provided in the packaging with the catheter 320. In other instances, the tip shaping tool 100, 200 may be provided separately and accessible during the medical procedure.

FIG. 9 depicts an illustrative graph 400 comparing angle retention between a distal tip region of a catheter shaped using a tip shaping tool in accordance with the disclosure versus a pre-shaped distal tip region of a catheter. A sample of catheters having a tip shaped with a tip shaping tool in accordance with this disclosure were compared to a sample of catheter having a pre-shaped tip. As discussed with reference to FIGS. 8A and 8B, the distal tip region of the catheter (e.g., distal tip region 321 of the catheter 320) may be formed with the catheter at room temperature at the location of the surgical procedure, without applying heat from a heating source. The distal tip region of the sample of catheters was shaped to a nominal angle of about 90 degrees with the tip shaping tool in accordance with this disclosure at room temperature. As shown in FIG. 9, the average initial tip angle 410 of the distal tip region of the sample catheters was about 90 degrees+/−2 degrees, as referenced at 415. The shaped distal tip region of the sample catheters was then held in a straightened configuration (a mandrel was inserted into the lumen to straighten the shaped distal tip to be parallel to the proximal catheter shaft) and submerged in a 37° Celsius water bath for thirty minutes. The sample catheters were then removed from the water bath and the angle of the distal tip region was again measured after removing the mandrel. As shown, the distal tip region of the sample catheters shaped in accordance with the disclosure retained about 84% of the initial angle 410, having a final angle 420 of about 76 degrees+/−2 degrees, as referenced at 425. Accordingly, the distal tip region of a catheter shaped in accordance with this disclosure has a distal tip angle retention of 60% or more, 70% or more, or 80% or more of its initial shaped angle.

In comparison, an initial angle of a sample of pre-shaped catheters was measured. The average initial angle 430 of a pre-shaped distal tip region of the sample of pre-shaped catheters was about 82 degrees+/−2 degrees, as referenced at 435. The sample of pre-shaped catheters was subjected to the same test. Namely, the shaped distal tip region of the sample of pre-shaped catheters were held in a straightened configuration (a mandrel was inserted into the lumen to straighten the pre-shaped distal tip to be parallel to the proximal catheter shaft) and submerged in a 37° Celsius water bath for thirty minutes. The sample pre-shaped tip catheters were then removed from the water bath and the angle of the distal tip region was again measured after removing the mandrel. The sample of pre-shaped distal tip region catheters had an average final angle 440 of about 54 degrees+/−2 degrees, as referenced at 445, and thus retained about 65% of the initial angle 430.

Thus, as shown in graph 400, the distal tip region shaped with a tip shaping tool in accordance with this disclosure has a better retention angle than a catheter having a pre-shaped distal tip region. The distal tip region retention allows a user (e.g., physician) to maintain access and/or navigate through tortuous vessels more readily throughout a medical procedure.

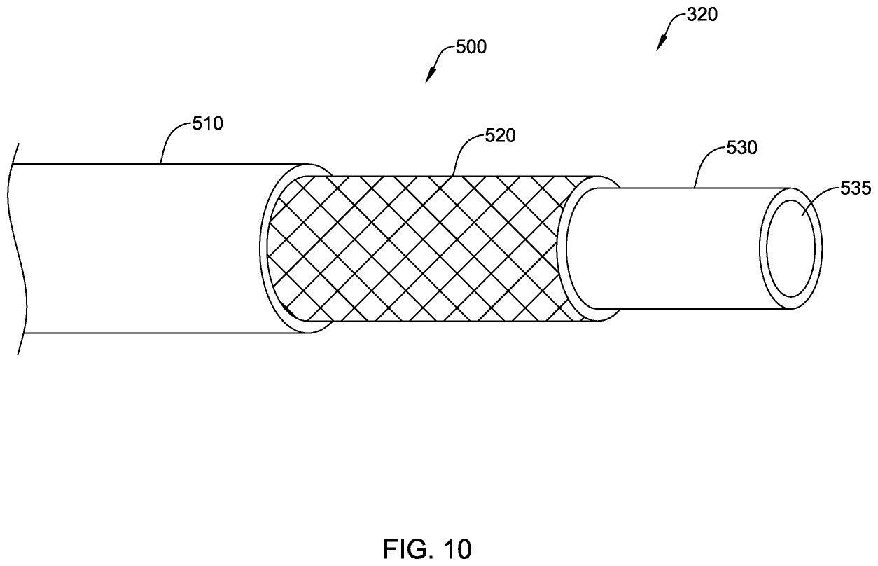

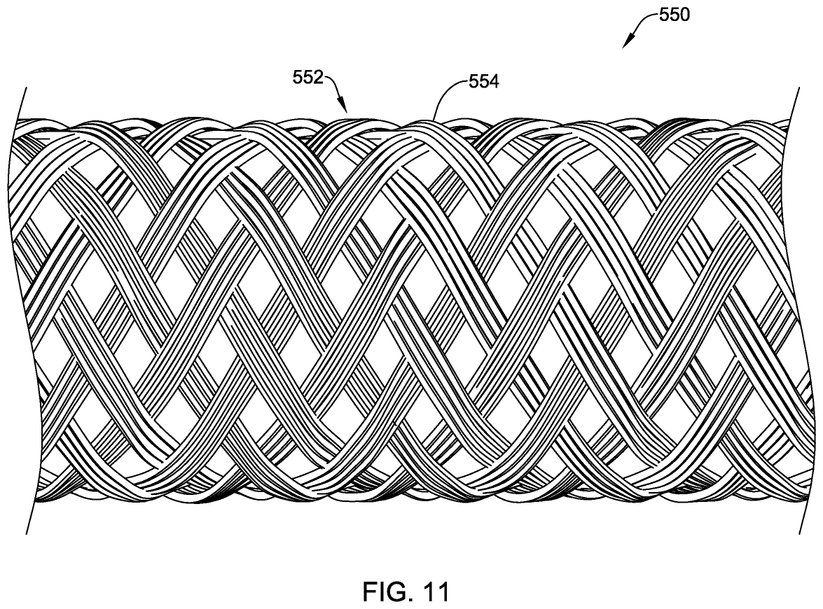

FIG. 10 is a partial view of an elongated shaft 500 of the catheter 320 in accordance with an embodiment of the disclosure, illustrating an outer layer 510, a middle layer, 520 and an inner layer 530. The inner layer 530 may define a lumen 535 extending therethrough. The lumen 535 may be considered as a guidewire lumen, as well as an injection lumen, or the like. As an example, in use, a practitioner may insert the catheter 320 over a guidewire (not shown). Once the target vessel is reached, the guidewire may be removed and a fluid may be injected at the target site through the lumen 535.

The inner layer 530 of the elongated shaft 500 may be formed from or include a coating of a material having a suitably low coefficient of friction. Examples of suitable materials may include polytetrafluoroethylene (PTFE). The inner layer 530 may be dimensioned to define the lumen 535, having an appropriate inner diameter to accommodate its intended use. In some cases, the inner layer 530 may define a lumen 535 having a diameter of about 0.021 inches to about 0.027 inches.

The outer layer 510 may be formed from a polymer that may provide the desired flexibility and strength. In some cases, the outer layer 510 may be formed from a nylon polymer, a thermoplastic polymer, elastomeric polyamides, or any other suitable polymer. The outer layer 510 may be dimensioned to define the outer diameter of the elongated shaft 500. In some cases, the outer diameter of the elongated shaft 500 may be less than 3 French. In some cases, the outer layer 510 may have an OD of 1.7 French or less, 2 French or less, 2.5 French or less, 2.8 French or less, or any other suitable outer diameter.

The middle layer 520 may be positioned between the outer layer 510 and the inner layer 530, and may be formed of a reinforcing structure, such as a braid or coil. The middle layer 520 may be considered to be a reinforcing layer that increases the torque response of the elongated shaft 500. The middle layer 520 may be formed of any suitable material, such as stainless steel, tungsten, gold, titanium, silver, copper, platinum, or nitinol. In some cases, the middle layer 520 may be formed from a non-metallic material such as polymer fibers, glass fibers, or liquid crystal polymer (LCP) fibers. The middle layer 520, when provide as a braided reinforcement layer, may be formed using a variety of different weave patterns, such as a three-over-three-under, a four-over-four-under, or the like. In some cases, the middle layer 520 may be formed using a two-over-two-under configuration, as will be discussed further with reference to FIG. 11.

FIG. 11 is a partial view of a braid 550 of the elongated shaft 500. As previously discussed, with reference to FIG. 9, the braid 550 may form the middle layer (e.g., middle layer 520) of the elongated shaft 500. The braid 550 may be formed from a plurality of strands 552, wherein each strand may be formed from a plurality of filaments 554. The braid 550 may comprise twelve (12) to twenty (20) strands 552. In some cases, the braid 550 may comprise sixteen (16) strands 552. The pattern of the braid 550 may be in the range of about 100 to about 140 picks per inch (PPI). In some cases, the pattern of the braid 550 may be in the range of about 120 PPI. In some examples, the braid 550 may achieve at least a seventy (70) percent coverage of the surface area of the outer surface of the inner layer 530, which thereby provides the elongated shaft 500 with a high burst pressure performance. In some cases, the braid 550 may achieve at least a sixty (60) percent coverage, an eighty (80) percent coverage, a ninety (90) percent coverage, or any other suitable percentage coverage of the surface area of the outer surface of the inner layer 530.

In some cases, as shown in FIG. 11, each of the plurality of strands 552 may include four filaments 554. The filaments 554 may be formed of any suitable material, such as stainless steel, tungsten, gold, titanium, silver, copper, platinum, or nitinol. In some cases, at least one strand 552 may include at least one stainless steel filament 554 and at least one tungsten filament 554. In other cases, at least one strand 552 may include two stainless steel filaments 554 and two tungsten filaments 554. In other cases, at least one strand 552 may be formed from four stainless steel filaments 554 and a second strand 552 may be formed from four tungsten filaments 554. Each strand 552 may be formed from any suitable combination of filaments 554, as desired. Some example strands may have five or more filaments. Other examples may have strands consisting of exactly four filaments.

In some cases, each strand 552 is formed from four filaments 554 arranged together side-by-side and braided in a two-over-two-under configuration. The four filament 554 braid 550 in a two-over-two-under configuration may provide the elongated shaft 500 with a greater torque response, and allow for greater pushability, thereby reducing the number of kinks that may form in the elongated shaft 500. The structure of the braid 550 may provide a desired radial strength such that the lumen 535 may not collapse during a catheter tip shaping process, such as the method disclosed herein. Further, the structure of the braid 550 may provide enough flexibility to allow a catheter tip, during the catheter tip shaping process to retain its desired shape (e.g., an invoked curvature).

The filaments 554 may have a round cross-sectional shape in some examples. Alternatively, the filaments 554 may include a flattened, rectangular, oval, or any other suitable cross-sectional shape. Each of the filaments 554 may include an outer diameter of less than 0.0009 inches. In some cases, each filament 554 may include an outer diameter of 0.00085 inches or less, 0.0008 inches or less, 0.00075 inches or less, 0.0006 inches or less, or any other suitable diameter. Filaments 554 may for example, have an outer diameter in the range of about 0.0006 inches to about 0.0009 inches, or in the range of about 0.00075 inches to about 0.00085 inches. Each filament 554 may be the same size and shape, or the filaments 554 may be of different sizes and shapes. For example, a strand 552 may include two larger tungsten filaments 554 and two smaller stainless-steel filaments 554.

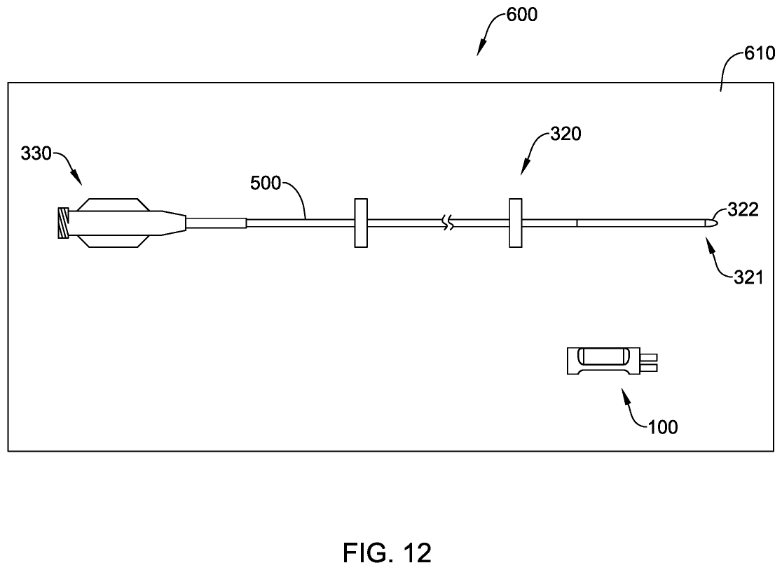

In some cases, a distal tip (shown in FIG. 12) may be provided at the distal end of the elongate shaft 500 of the catheter 320. The distal tip may be a polymeric distal tip, which may be formed from an elastomer (e.g., Pebax®), a thermoplastic polymer, or any other suitable polymer. The distal tip may be formed of a softer material than other portions of the elongate shaft 500 of the catheter 320, such as by using a polymer or elastomer having a shore hardness of less than 63D. In some cases, the distal tip may be formed from a polymer with a shore hardness of about 40D or 35D. In some cases, the distal tip may have a length of about 1 millimeter (mm). In other cases, the distal tip may have a length of about 1.5 mm, about 1.3 mm, about 1.7 mm, or any other suitable length.

The elongated shaft of the catheter 320 may be adapted to provide enhanced torque response, meaning that a particular rotation made at the proximal end region will be communicated to the distal end region 321. In some cases, the elongated shaft may be adapted to provide sufficient torque response such that a particular rotation made at the proximal end region will provide a torque response of at least 0.9:1 (e.g., ninety (90) percent) at the distal end region 321. As an example, a 90-degree rotation made at the proximal end region will correlate to a rotation made at the distal end region 321 that is about 81 degrees. In some cases, the elongated shaft may provide a torque response of at least 0.95:1 (e.g., ninety-five (95) percent). As an example, a 90-degree rotation made at the proximal end region will correlate to a rotation made at the distal end region 321 that is about 85.5 degrees. In other cases, the elongated shaft may provide a torque response of at least 0.98:1 (e.g., ninety-eight (98) percent) at the distal end region 321. As an example, a 360-degree rotation made at the proximal end region will correlate to a rotation made at the distal end region 321 that is around 356 degrees. These are just examples.

FIG. 12 depicts a kit 600 including a catheter 320 and a tip shaping tool 100 provided in packaging 610 in which the catheter 320 and the tip shaping tool 100 are provided in. As shown in FIG. 12, the catheter 320 may include an elongated shaft 500 having a distal end region 321 extending to the distalmost tip 322. The catheter 320 may include a hub 330 secured to a proximal end of the elongate shaft 500. The catheter 320 may have any desired length and outer diameter. For example, the catheter 320 may have a length that is in the range of about 50 to 200 centimeters and may have an outer diameter (OD) that is less than 3 French, for example. In some cases, the catheter 320 may have an OD of 1.7 French or less, 2 French or less, 2.5 French or less, 2.8 French or less, or any other suitable outer diameter. In some cases, the catheter 320 may include an OD of about 2.6 French along the distal end region 321, and an OD of about 2.8 French along the proximal end region. In some cases, the catheter 320 may have an inner diameter (ID) of about 0.021 inches to 0.027 inches, for example. In other cases, the catheter 610 may have an ID of about 0.02 inches, 0.03 inches, or any other suitable inner diameter. These sizes may vary depending on particular usage.

Although the catheter 320 is shown packaged with the tip shaping tool 100, in other instances the catheter 320 can be packaged in the packaging 610 with the tip shaping tool 200, or another tip shaping tool having features and functionality as disclosed herein. Accordingly, when opening the packaging 610 to access the catheter 320, the medical personnel may also be provided with the tip shaping tool 100, 200 to intra-operatively manually shape the distal end region of the catheter 320 to a desired curvature or shape. These are just examples. The tip shaping tool 100, 200, the catheter 320, and various components thereof, may be manufactured according to essentially any suitable manufacturing technique including extruding, co-extruding, molding, casting, 3-D printing, mechanical working, and the like, or any other suitable technique. Furthermore, the various structures may include materials commonly associated with medical devices such as metals, metal alloys, polymers (some examples of which are disclosed below), metal-polymer composites, ceramics, combinations thereof, and the like, or other suitable material. These materials may include transparent or translucent materials to aid in visualization during the procedure. Some examples of suitable metals and metal alloys includes stainless steel, such as 304V, 304L, and 316LV stainless steel, mild steel, nickel-based alloys, or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In some embodiments the sheath can be blended with a liquid crystal polymer (LCP). For example, the mixture can contain up to about 6 percent LCP.

In at least some embodiments, portions or all of the catheter 320 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the catheter 610 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the catheter 320 to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A catheter tip shaping system comprising:
a catheter; and
a tip shaping tool, comprising:
a body having a first end and a second end;
a first pin, a second pin, and a third pin, wherein the first, second, and third pins are positioned at the first end of the body and extend from a first face of the body;
wherein a distal end region of the catheter is configured to be positioned between a gap between the first, second, and third pins such that the tip shaping tool can be manipulated by a user to invoke a curvature in the distal end region of the catheter.

2. The catheter tip shaping system of claim 1, wherein the first, second, and third pins are positioned equidistant from one another.

3. The catheter tip shaping system of claim 2, wherein a distance between each of the first, second, and third pins is about 0.025 inches to about 0.045 inches.

4. The catheter tip shaping system of claim 1, wherein the body includes a grip portion to be grasped by the user.

5. The catheter tip shaping system of claim 1, wherein the first, second, and third pins each include a circular cross-section.

6. The catheter tip shaping system of claim 1, wherein the first, second, and third pins each include a first end region and a second end region, wherein the first end region includes a first outer diameter and the second end region includes a second outer diameter, and the first outer diameter is greater than the second outer diameter.

7. The catheter tip shaping system of claim 6, wherein the second end regions of the first, second, and third pins are coupled to the first end of the body of the tip shaping tool with the second end regions of the first, second, and third pins positioned between the first face of the body and the first end regions of the first, second and third pins.

8. The catheter tip shaping system of claim 7, wherein a length of the second end regions of the first, second, and third pins is greater than an outer diameter of the distal end region of the catheter.

9. The catheter tip shaping system of claim 7, wherein a gap between the first end regions of the first, second, and third pins is less than an outer diameter of the distal end region of the catheter and a gap between the second end regions of the first, second, and third pins is greater than the outer diameter of the distal end region of the catheter.

10. The catheter tip shaping system of claim 9, wherein the first pin is positioned on a first side of the distal end region of the catheter and the second and third pins are positioned on an opposite, second side of the distal end region of the catheter when the distal end region of the catheter is positioned in the gap.

11. The catheter tip shaping system of claim 10, wherein the body is configured to be rotated to invoke the curve in the distal end region.

12. The catheter tip shaping system of claim 11, wherein the first pin is located on a concave side of the curve, and the second and third pins are located on a convex side of the curve.

13. The catheter tip shaping system of claim 1, wherein the catheter comprises:
an elongated shaft having a distal end and a proximal end, the elongated shaft including:
an outer layer formed of a polymer;
an inner layer formed of a polymer; and
a middle layer formed of a braid having a plurality of strands, each strand including a plurality of metal filaments and a polymeric distal tip attached to the distal end of the elongated shaft.

14. A method of shaping a distal end of a catheter using a tip shaping tool, the method comprising:
inserting a distal end region of the catheter in a gap between a first, a second, and a third pin of the tip shaping tool, the tip shaping tool including:
a body having a first end and a second end;
wherein the first pin, the second pin, and the third pin are positioned at the first end of the body and extend from a first face of the body;
rotating the tip shaping tool such that the distal end region of the catheter moves from a first, straight configuration to a second, curved configuration; and
removing the distal end region of the catheter from the tip shaping tool;
wherein the tip shaping tool can be rotated in a clockwise direction and/or a counter-clockwise position.

15. The method of claim 14, wherein when the distal end of the catheter is inserted into the gap of the tip shaping tool, the first pin is located on a first side of the distal end region of the catheter, and the second and third pins are positioned on an opposite, second side of the distal end region of the catheter.

16. The method of claim 14, wherein the first pin is located on a concave side of the distal end region in the second, curved configuration, and the second and third pins are located on a convex side of the distal end region in the second, curved configuration.

17. The method of claim 14, wherein when the distal end region of the catheter is inserted into the tip shaping tool, an end of a marker band positioned on the catheter is aligned with an edge of the first end of the tip shaping tool prior to rotating the tip shaping tool.

18. A catheter tip shaping tool comprising:
a body having a first end and a second end; and
a first pin, a second pin, and a third pin, wherein the first, second, and third pins are positioned at the first end of the body and extend from a first face of the body.

19. The catheter tip shaping tool of claim 18, wherein the first, second, and third pins are positioned equidistant from one another.

20. The catheter tip shaping tool of claim 18, wherein the first, second, and third pins each include a first end region and a second end region, wherein the first end region is positioned between the first face of the body and the second end region, and the first end region includes a smaller outer diameter than an outer diameter of the second end region.
